(19) United States
(12) Patent Application Publication
(10) Pub. No.: US 2023/0046914 A1
(43) Pub. Date: Feb. 16, 2023

(54) HASHISH PRODUCT HAVING ENHANCED USER EXPERIENCE AND MANUFACTURING PROCEDURE FOR OBTAINING SAME

(71) Applicant: HEXO OPERATIONS INC., Ottawa (CA)

(72) Inventors: Brendan Kinahan, Ottawa (CA); George Elvira, Montreal (CA); Denis Keseris, Baie d'Urfe (CA); Renato Devien Durbano, Ottawa (CA)

(21) Appl. No.: 17/786,506

(22) PCT Filed: Dec. 16, 2020
(86) PCT No.: PCT/CA2020/051733

§ 371 Date: Jun. 16, 2022

Related U.S. Application Data
(60) Provisional application No. 62/948,576, filed on Dec. 16, 2019.

(51) Int. Cl. A24B 15/16; A24B 15/30

(52) U.S. Cl. CPC A24B 15/16; A24B 15/303

(57) ABSTRACT

The present disclosure relates to a hashish product having improved consistency, texture and/or appearance. For example, the hashish product may include a cohesive mass of isolated trichomes and a moisture content that is distributed throughout in the cohesive mass. The present disclosure also relates to a process for making the hashish product which includes incorporating water in a mass of isolated trichomes prior to pressing same.

### CROSS-REFERENCE TO RELATED APPLICATION

The present application claims the benefit of U.S. provisional patent application Ser. No. 62/948,576 filed on Dec. 16, 2019. The contents of the above-referenced document are incorporated herein by reference in their entirety.

### TECHNICAL FIELD

This application generally relates to the field of cannabis-based consumer products and, more specifically, to hashish products as well as methods of manufacturing same.

### BACKGROUND

With stage-wise legalization of cannabis-based consumer products in Canada and eventually in various other areas in the world, advancements in extraction technology, industrial scale production and accessibility to a wide variety of forms have accelerated in order to fulfill emerging demands.

Hashish (or hash) is a concentrated derivative of the dried resin glands, known as trichomes, of mature and unpollinated female cannabis plants. Hash contains the same active ingredients as marijuana—including cannabinoids such as tetrahydrocannabinol and others—although at higher concentrations than the un-sifted buds or leaves from which dried marijuana is made, which leads to higher potency. The trichomes may be removed from the plant material by mechanical or chemical means.

Separated trichomes have a powder appearance (referred to as “kief”) and pressed to obtain blocks of hash, the color and pliability of which can vary widely based on the source material, the extraction method, and the production conditions. For example, dry-sift pressed hashish is usually solid, whereas water-purified hashish—often called bubble hashish—can be a paste-like substance with varying cohesion and stickiness in addition to being in powder form or pressed into bricks. The color of a hashish product is most commonly light to dark brown, but can also vary from transparent to yellow, tan, black, or red.

Hand or mechanical presses are often used to produce hash products. However, hand presses are too small and inefficient for commercial volume production, while mechanical presses lead to variability of the finished hash product and an inconsistent product batch-over-batch. Furthermore, obtaining the desirable pliability and hardness requires a significant amount of “art” that is hardly reproducible and the skills of the individual play a key role in defining the quality of the finished product—characteristics that are undesirable when designing and implementing industrial scale procedures.

Considering the above, it would be highly desirable to be provided with a hashish product and systems or methods for making same that would at least partially alleviate some of the disadvantages of the existing technologies.

### SUMMARY

This Summary is provided to introduce a selection of concepts in a simplified form that are further described below in the Detailed Description. This Summary is not intended to identify key aspects or essential aspects of the claimed subject matter.

As embodied and broadly described herein, the present disclosure relates to a hashish product, comprising a cohesive mass of isolated cannabis trichomes and at least 5 wt. % moisture content, wherein the moisture content is distributed throughout the cohesive mass.

Implementations of this hashish product can include one or more of the following features:
comprising from 5 wt. % to about 10 wt. % moisture content.
comprising at least 7.5 wt. % moisture content.
comprising at least 10 wt. % moisture content.
comprising less than 50 wt. % moisture content.
comprising less than 40 wt. % moisture content.
comprising less than 35 wt. % moisture content.
comprising less than 30 wt. % moisture content.
comprising from about 15 wt. % to about 35 wt. % moisture content.
comprising less than 1000 ppm of an organic solvent, including 0.
comprising less than 500 ppm of an organic solvent, including 0.
the moisture content is detected in at least 90 vol. %, or in at least 95 vol. %, or in at least 99 vol. %, or in 100% of the cohesive mass.
the hashish product has a weight of from about 0.2 g to about 20 g.
the hashish product comprises a cannabinoid selected from tetrahydrocannabinol (THC), cannabidiol (CBD), cannabigerol (CBG), cannabinol (CBN), and any combinations thereof.
comprising a cannabinoid content of at least 20 wt. %.
the cannabinoid content is of at least 25 wt. %.
the isolated cannabis trichomes prior to forming the cohesive mass are kief.
the kief is a dry-sifted kief.
the kief is from one or more kief grade.
the kief is a low grade kief comprising a tetrahydrocannabinol (THC) content of less than 40 wt. %.
the isolated cannabis trichomes are from one or more strain(s) of cannabis plant.

As embodied and broadly described herein, the present disclosure relates to a process of making a hashish product, comprising a) providing isolated cannabis trichomes; b) incorporating water into the batch of isolated cannabis trichomes and mixing same to obtain a mixture; and c) pressing said mixture to obtain the hashish product, the hashish product comprising a cohesive mass of the isolated cannabis trichomes, wherein the water is distributed throughout the cohesive mass.

Implementations of this process can include one or more of the following features:
water at step b) is incorporated in the form of liquid, steam, or a combination thereof.
said water is distilled water, reverse osmosis water, or microfiltered water.
water at step b) is incorporated to obtain at least 5 wt. % moisture content.
water at step b) is incorporated to obtain at least 10 wt. % moisture content.
water at step b) is incorporated to obtain from 5 wt. % to about 10 wt. % moisture content.
water at step b) is incorporated to obtain at least 7.5 wt. % moisture content.
water at step b) is incorporated to obtain at least 10 wt. % moisture content.
water at step b) is incorporated to obtain less than 50 wt. % moisture content.
water at step b) is incorporated to obtain less than 40 wt. % moisture content.
water at step b) is incorporated to obtain less than 35 wt. % moisture content.
water at step b) is incorporated to obtain less than 30 wt. % moisture content.
water at step b) is incorporated to obtain from about 15 wt. % to about 35 wt. % moisture content.
said pressing at step c) is performed with a pressure of from about 0.5 up to about 15 US tons.
the pressure is from about 1 up to about 10 US tons.
the pressure is from about 1 up to about 5 US tons.
said pressing at step c) is performed for a duration of up to about 5 minutes.
said pressing at step c) is performed for a duration of up to about 3 minutes.
further comprising heating while mixing.
the heating is performed with a temperature of from about 30° C. to about 120° C.
the heating is performed with a temperature sufficient to decarboxylate at least a portion of one or more cannabinoids in the batch of isolated cannabis trichomes.
the heating is performed with a temperature of less than 120° C.
the temperature is of less than 90° C.
the temperature is of less than 85° C.
the temperature is of at least 80° C.
the step c) comprises placing the mixture in a mold and pressing the mixture with a press.
the hashish product comprises a cannabinoid selected from tetrahydrocannabinol (THC), cannabidiol (CBD), cannabigerol (CBG), cannabinol (CBN), and any combinations thereof.
the hashish product comprises a cannabinoid content of at least 20 wt. %.
the cannabinoid content is of at least 25 wt. %.
the isolated cannabis trichomes in the step a) are kief.
the kief is dry-sifted kief
the kief is from one or more kief grade.
the kief is a low grade kief comprising a tetrahydrocannabinol (THC) content of less than 40 wt. %.
the isolated cannabis trichomes are from one or more strain(s) of cannabis plant.
further comprising processing the hashish product to obtain a hashish product unit having a weight of from about 0.2 g to about 20 g.
the moisture content is detected in at least 90 vol. %, or in at least 95 vol. %, or in at least 99 vol. %, or in 100% of the hashish product.

As embodied and broadly described herein, the present disclosure relates to a hashish product made by the process described above.

As embodied and broadly described herein, the present disclosure relates to a hashish product comprising a cohesive mass of isolated trichomes, the product having at least two of the following properties as determined in a three-point bending test: a breaking point property of at least 250 g; a stiffness property of less than 8,500 g/mm; and a toughness property of less than 12,500 g*mm, wherein the isolated trichomes prior to forming the cohesive mass are low grade kief comprising a tetrahydrocannabinol (THC) content of less than 40 wt. %.

Implementations of this hashish product can include one or more of the following features:
further comprises a cannabinoid selected from cannabidiol (CBD), cannabigerol (CBG), cannabinol (CBN), and any combinations thereof.
comprising a cannabinoid content of at least 20 wt. %.
the cannabinoid content is of at least 25 wt. %.
the kief is a dry-sifted kief.
the kief is from one or more kief grade.
the isolated cannabis trichomes are from one or more strain(s) of cannabis plant.
the breaking point is from about 500 g to about 2500 g.
the stiffness is from about 200 g/mm to about 1200 g/mm.
the toughness is from about 1500 g*mm to about 12,000 g*mm.

All features of exemplary embodiments which are described in this disclosure and are not mutually exclusive can be combined with one another. Elements of one embodiment can be utilized in the other embodiments without further mention. Other aspects and features of the present invention will become apparent to those ordinarily skilled in the art upon review of the following description of specific embodiments in conjunction with the accompanying Figures.

### BRIEF DESCRIPTION OF THE DRAWINGS

A detailed description of specific exemplary embodiments is provided herein below with reference to the accompanying drawings in which:

FIG. 1 illustrates a non-limiting flowchart example of a process for making a hashish product in accordance with an embodiment of the present disclosure;

FIG. 2 illustrates a non-limiting flowchart example of a process for pressing trichomes for making a hashish product in accordance with an embodiment of the present disclosure;

FIG. 3 illustrates a non-limiting flowchart example of post-pressing steps for making hashish product in accordance with an embodiment of the present disclosure;

FIG. 4 illustrates a non-limiting flowchart example of a process for assessing quality of the hashish product in accordance with an embodiment of the present disclosure;

FIG. 5A-5C are non-limiting schematic representations of examples of a quality test where samples are taken from a hashish block.

FIG. 6 is a non-limiting graph representing results obtained from a three-point bending test on a hashish sample of the present disclosure.

In the drawings, exemplary embodiments are illustrated by way of example. It is to be expressly understood that the description and drawings are only for the purpose of illustrating certain embodiments and are an aid for understanding. They are not intended to be a definition of the limits of the invention.

### DETAILED DESCRIPTION

A detailed description of one or more embodiments of the invention is provided below along with accompanying figures that illustrate the principles of the invention. The invention is described in connection with such embodiments, but the invention is not limited to any embodiment. The scope of the invention is limited only by the claims. Numerous specific details are set forth in the following description to provide a thorough understanding of the invention. These details are provided for the purpose of non-limiting examples and the invention may be practiced according to the claims without some or all these specific details. Technical material that is known in the technical fields related to the invention has not been described in detail so that the invention is not unnecessarily obscured.

The present inventors have surprisingly and unexpectedly discovered that at least some of the problems discussed above with respect to lack of reproducible industrial procedures for making hashish products of consistent and sufficient quality can be resolved with the herein described procedures.

While various references suggest completely drying isolated cannabis trichomes prior to making hash based on the belief that fully drying cannabis trichomes avoid eventual mold formation, the present inventors have surprisingly discovered that incorporating water into the isolated cannabis trichomes prior to pressing same is beneficial from the perspective of obtaining consistent and sufficient quality. Furthermore, the present inventors have discovered that mold formation is virtually non-existent when moisture content of the hashish is fine-tuned to obtain the contents described herein.

For example, the present inventors using prior art procedures have produced hashish products that, when bone-dry, lack textural consistency and pliability, which often creates a substantial amount of fine crumbles during handling. Such lack of textural consistency, pliability and/or crumbliness may result in a suboptimal user experience as it renders handling a hashish product more difficult and results in undesirable waste when the user segments a hashish product to obtain a usable portion. Segmentation of a hashish product occurs when a user handles the product in order to consume same. Users can, for example, simply crumble a segment of a hashish product and add same into a joint, alone or mixed with tobacco or marijuana buds. User can also place a segment of the hashish product in a pipe or bong for smoking hash. Another method of consuming may involve dabbing a segment of the hashish product or using hot knives sandwiching the segment of the hashish product, where the heat from the pre-heated knives produces smoke which is inhaled by the user. Yet another method involves using a vaporizer (or pen) in which the user places the segment of the hashish product.

Furthermore, the present inventors have discovered that another source of quality variation in the production of hash is the composition of the kief. Kief containing higher amounts of plant matter and/or lower amounts of cannabinoids yield a product that is crumbly and does not have a level of cohesion or stickiness that allows for formation of bricks. This leads to a less-than-desirable product that may provide for inconsistent and less enjoyable user experience.

Without being bound by any theory, it is believed that the herein described hashish product may provide advantageous benefits. For example, the herein described hashish product may have improved textural consistency, pliability and/or crumbliness characteristics. One or more of these characteristics may lead to better level of cohesion or stickiness that allows for formation of bricks as well as to better segmentation during use thereby resulting in reduction of waste material during use.

Without being bound by any theory, it is believed that the herein described hashish manufacturing methods may also provide advantageous benefits. For example, the herein described manufacturing methods may result in substantially fewer quality failures (e.g., based on textural consistency, pliability and/or crumbliness). Additionally, or alternatively, the herein described manufacturing methods may result in reduction of waste materials during manufacturing of hashish products as these methods allow for a wider range of input materials to be selected, thereby reducing the amount of plant waste that is otherwise discarded. Additionally, or alternatively, the herein described manufacturing methods may result in more manufacturing flexibility and/or improved inventory management in terms of allowing use of various grades of kief and/or various cannabis plant strains. The reader will recognize that one or more of these benefits are advantageous in the context of large-scale industrial production.

### Hashish Products

The hashish product of the present disclosure comprises a cohesive mass of isolated cannabis trichomes.

As used herein, the term “cannabis trichomes” generally refers to crystal-shaped outgrowths or appendages (also called resin glands) on cannabis plants typically covering the leaves and buds. Trichomes produce hundreds of known cannabinoids, terpenes, and flavonoids that make cannabis strains potent, unique, and effective.

As used herein, the term “cannabis plant(s)”, encompasses wild type Cannabis (including but not limited to the species Cannabis sativa, Cannabis indica and Cannabis ruderalis) and also variants thereof, including cannabis chemovars (or “strains”) that naturally contain different amounts of the individual cannabinoids. For example, some Cannabis strains have been bred to produce minimal levels of tetrahydrocannabinol (THC), the principal psychoactive constituent responsible for the high associated with it and other strains have been selectively bred to produce high levels of THC and other psychoactive cannabinoids. Cannabis plants produce a unique family of terpeno-phenolic compounds called cannabinoids, some of which produce the “high” one experiences from consuming marijuana.

As used herein, the term “isolated cannabis trichomes” refers to trichomes that have been separated from cannabis plant material plant using any method known in the art. For example, and without wishing to be limiting in any manner, the isolated cannabis trichomes may be obtained by a chemical separation method or may be separated by dry sifting, by sieving through a screen by hand or in motorized tumblers (see for example WO 2019/161509). The details of various methods for separating trichomes from the cannabis plant are well-known in the art. Isolated cannabis trichomes obtained by separation of trichomes from the cannabis plant material is typically referred to as “kief” (also “keef” or “kif”) and has a powdery appearance. Because of inherent limitations to existing separation methods, some plant matter or other foreign matter can be present in isolated cannabis trichomes. Preferably, the trichome separation technique of the present disclosure does not involve contacting the trichomes with water.

In some embodiments, the isolated cannabis trichomes forming the hashish product of the present disclosure may originate from one or more than one strain of cannabis plant. It is known amongst consumers of hashish and other cannabis products that using isolated cannabis trichomes produced from more than one strain of cannabis plant allows a user to tune the psychoactive and/or entourage effect obtained by consuming the product. The mixing of cannabis plant strains may also allow adjustment of the final concentration of a component of the product, for example but not limited to the cannabinoid content. Additionally, use of more than one strain allows for improved product and waste management—important in commercial production.

In some embodiments, the isolated cannabis trichomes forming the hashish product of the present disclosure may originate from one or more kief grades.

As used herein, the term “kief grade” refers to quantitative and/or qualitative characteristics of the purity of isolated trichomes. For example, this term may provide information relative to the cannabinoid content and/or residual plant matter content in the kief.

For example, low grade kief made by dry sifting may include a mixture of trichome heads, stalks, and plant material (contaminate) and will char (the opposite of melt) immediately upon being dabbed. Low grade kief generally have lower than 40 wt. % THC content. Medium grade kief made by dry sifting will typically contain less plant material contaminants than low grade kief and will generally include from 40 wt. % to 60 wt. % THC. High grade kief made by dry sifting contains substantially only pure isolated trichome heads and will generally include more than 60 wt. % THC.

In some embodiments, the hashish product of the present disclosure comprises a cohesive mass of isolated trichomes and a moisture content (also referred as “water content”) that is distributed throughout the cohesive mass.

As used herein, the term “distributed throughout” with respect to the moisture content means that the moisture content is detectable, for example, in at least 80 vol. %, or in at least 85 vol. %, or in at least 90 vol. %, or in at least 95 vol. % of the hashish product.

The moisture content can be detected and quantified using quantitative methods. For example, and without wishing to be limiting in any manner, the moisture content may be quantified using any suitable technique, such as for example Gas Chromatography/Mass Spectrometry (GC/MS), High Pressure Liquid Chromatography (HPLC), Gas Chromatography/Flame Ionization Detection (GC/FID), infra-red spectrum (IR) spectroscopy, ultra-violet spectrum (UV) spectroscopy, Raman spectroscopy, and the like. Other techniques may be used, for example by heating a test sample to a temperature near water ebullition point (100° C.) for a given time sufficient to evaporate substantially all water contained in the sample and then measuring the sample weight loss that corresponds to the moisture content initially present in the sample. Other techniques may involve measuring water activity, for example using a capacitive hygrometer (e.g., the Aqualab™ 4TE (Meter, USA)) using the chilled-mirror dew point technique, or may involve measuring water content, for example using a moisture analyzer (e.g., MA160 Infrared Moisture Analyzer (Sartorius AG, Germany) using the loss on drying technique.

In some embodiments, the moisture content in the hashish product of the present disclosure, achieved through addition of water to the kief, can be of at least 5 wt. %. For example, the moisture content can be of from 5 wt. % to about 10 wt. %. For example, the moisture content can be of at least 7.5 wt. % moisture content, or at least 10 wt. %. For example, the moisture content can be of less than 50 wt. %, less than 40 wt. %, less than 35 wt. %, less than 30 wt. % moisture content. For example, the moisture content can be of from 10 wt. % to about 50 wt. %, including any ranges therein or any values therein, such as from about 15 wt. % to about 35 wt. % moisture content. The reader will understand that these percentages are expressed as weight of the water/total weight of the cohesive mass containing the water. Further, while higher moisture content than 50 wt. % have been made by the present inventors, it has been observed that such products may be more difficult to remain mold-free over time. For example, the moisture content can be of about 7.5%, about 10 wt. %, about 15 wt. %, about 20 wt. %, about 25 wt. %, about 30 wt. %, about 35 wt. %, about 40 wt. %, or about 45 wt. %, or any ranges with any of these values as range limits.

Without being bound by any particular theory, it is believed that the herein described addition of water during the manufacturing of hashish products leads to the aforementioned moisture content distribution in the hashish product, which alone or together with various other cannabis components present in the cannabis trichomes (e.g., waxes, terpenes, cannabinoids) during the isolated trichomes pressing affords the herein described observed improvement in the textural consistency, pliability and/or crumbliness of the hashish product and associated better user experience relative to those of a similar product but with lower moisture content.

In some embodiments, the textural consistency, pliability and/or crumbliness of the hashish product can be characterized with material properties of the product, for example as determined with a three-point bending test (e.g., using a Texture Analyzer). In such embodiments, the hashish product can be characterized as having at least one or more of the following properties: a breaking point of at least about 250 g, a stiffness of less than about 8500 g/mm, and a toughness of less than about 12,500 g*mm.

For example, the breaking point can be of from about 250 g to about 8000 g, including any ranges there in-between or any values therein. For example, the breaking point can be of about 250 g, about 500 g, about 750 g, about 1000 g, about 1150 g, about 1200 g, about 1250 g, about 1300 g, about 1350 g, about 1400 g, about 1450 g, about 1500 g, about 1550 g, about 1600 g, about 1650 g, about 1700 g, about 1750 g, about 1800 g, about 1850 g, about 1900 g, about 1950 g, about 2000 g, about 2500 g, about 3500 g, about 4500 g, about 5500 g, about 6500 g, about 7500 g, or about 8000 g, including any value there in-between or any ranges with any of these values as range limits. Preferably, the breaking point is from about 500 g to about 2500 g.

For example, the stiffness can be of from about 200 g/mm to about 8500 g/mm, including any ranges there in-between or any values therein. For example, the stiffness can be of about 200 g/mm, about 250 g/mm, about 300 g/mm, about 350 g/mm, about 400 g/mm, about 450 g/mm, 500 g/mm, about 550 g/mm, about 600 g/mm, about 650 g/mm, about 700 g/mm, about 750 g/mm, about 800 g/mm, about 850 g/mm, about 900 g/mm, about 950 g/mm, about 1000 g/mm, about 1050 g/mm, about 1100 g/mm, about 1150 g/mm, or about 1200 g/mm, including any value there in-between or any ranges with any of these values as range limits. Preferably, the stiffness is from about 200 g/mm to about 1200 g/mm.

For example, the toughness can be of from about 1500 g*mm to about 12,500 g*mm, including any ranges there in-between or any values therein. For example, the toughness can be of about 1500 g*mm, about 2500 g*mm, about 3500 g*mm, about 4500 g*mm, about 5500 g*mm, about 6500 g*mm, about 7500 g*mm, about 8500 g*mm, about 9500 g*mm, about 10,500 g*mm, about 11,500 g*mm, or about 12,000 g*mm, including any value there in-between or any ranges with any of these values as range limits. Preferably, the toughness is from about 1500 g*mm to about 12,000 g*mm.

In some embodiments, the hashish product of the present disclosure is made without (or with minimal) use of organic solvents. Accordingly, the hashish product of the present disclosure is substantially free from organic solvents. For example, the hashish product may include less than 1000 ppm, or less than 500 ppm of an organic solvent, including 0. Such virtual absence of an organic solvent may be advantageous to meet market demand for more health-centric products, in which the presence of cancer-causing organic solvents is to be avoided.

The hashish product of the present disclosure contains one or more cannabinoid.

As used herein, the term “cannabinoid” generally refers to chemical compounds that act upon a cannabinoid receptor such as CB1 and CB2 and that are found in cannabis plants. Examples of cannabinoids include, but are not limited to, cannabichromanon (CBCN), cannabichromene (CBC), cannabichromevarin (CBCV), cannabicitran (CBT), cannabicyclol (CBL), cannabicyclovarin (CBLV), cannabidiol (CBD, defined below), cannabidiol monomethylether (CBDM), cannabidiol-C4 (CBD-C4), cannabidiorcol (CBD-C1), cannabidiphorol (CBDP), cannabidivarin (CBDV), cannabielsoin (CBE), cannabifuran (CBF), cannabigerol (CBG), cannabigerol monomethylether (CBGM), cannabigerolic acid (CBGA), cannabigerovarin (CBGV), cannabinodiol (CBND), cannabinodivarin (CBVD), cannabinol (CBN), cannabinol methylether (CBNM), cannabinol propyl variant (CBNV), cannabinol-C2 (CBN-C2), cannabinol-C4 (CBN-C4), cannabiorcol (CBN-C1), cannabiripsol (CBR), cannabitriol (CBO), cannabitriolvarin (CBTV), cannabivarin (CBV), dehydrocannabifuran (DCBF), Δ7-cis-iso tetrahydrocannabivarin, tetrahydrocannabinol (THC, defined below), Δ9-tetrahydrocannabinolic acid A (THCA-A), Δ9-tetrahydrocannabionolic acid B (THCA-B), Δ9-tetrahydrocannabiorcol (THC-C1), tetrahydrocannabivarinic acid (THCVA), tetrahydrocannabivarin (THCV), ethoxy-cannabitriolvarin (CBTVE), trihydroxy-Δ9-tetrahydrocannabinol (triOH-THC), 10-ethoxy-9hydroxy-Δ6a-tetrahydrocannabinol, 8,9-dihydroxy-Δ6a-tetrahydrocannabinol, 10-oxo-Δ6a-tetrahydrocannabionol (OTHC), 3,4,5,6-tetrahydro-7-hydroxy-α-α-2-trimethyl-9-n-propyl-2, 6-methano-2H-1-benzoxocin-5-methanol (OH-iso-HHCV), Δ6a,10a-tetrahydrocannabinol (Δ6a,10a-THC), Δ8-tetrahydrocannabivarin (Δ8-THCV), Δ9-tetrahydrocannabiphorol (Δ9-THCP), Δ9-tetrahydrocannabutol (Δ9-THCB), derivatives of any thereof, and combinations thereof. Further examples of cannabinoids are discussed in at least WO2017/190249 and U.S. Patent Application Pub. No. US2014/0271940, which are each incorporated by reference herein in their entirety.

Cannabidiol (CBD) means one or more of the following compounds: Δ2-cannabidiol, Δ5-cannabidiol (2-(6-isopropenyl-3-methyl-5-cyclohexen-1-yl)-5-pentyl-1,3-benzenediol); Δ4-cannabidiol (2-(6-isopropenyl-3-methyl-4-cyclohexen-1-yl)-5-pentyl-1,3-benzenediol); Δ3-cannabidiol (2-(6-isopropenyl-3-methyl-3-cyclohexen-1-yl)-5-pentyl-1,3-benzenediol); Δ3,7-cannabidiol (2-(6-isopropenyl-3-methylenecyclohex-1-yl)-5-pentyl-1,3-benzenediol); Δ2-cannabidiol (2-(6-isopropenyl-3-methyl-2-cyclohexen-1-yl)-5-pentyl-1,3-benzenediol); Δ1-cannabidiol (2-(6-isopropenyl-3-methyl-1-cyclohexen-1-yl)-5-pentyl-1,3-benzenediol); and Δ6-cannabidiol (2-(6-isopropenyl-3-methyl-6-cyclohexen-1-yl)-5-pentyl-1,3-benzenediol). In a preferred embodiment, and unless otherwise stated, CBD means Δ2-cannabidiol.

Tetrahydrocannabinol (THC) means one or more of the following compounds: Δ8-tetrahydrocannabinol (Δ8-THC), Δ9-cis-tetrahydrocannabinol (cis-THC), Δ9-tetrahydrocannabinol (Δ9-THC), Δ10-tetrahydrocannabinol (Δ10-THC), Δ9-tetrahydrocannabinol-C4 (THC-C4), Δ9-tetrahydrocannabinolic acid-C4 (THCA-C4), synhexyl (n-hexyl-Δ3THC). In a preferred embodiment, and unless otherwise stated, THC means one or more of the following compounds: Δ9-tetrahydrocannabinol and Δ8-tetrahydrocannabinol.

A cannabinoid may be in an acid form or a non-acid form, the latter also being referred to as the decarboxylated form since the non-acid form can be generated by decarboxylating the acid form. Within the context of the present disclosure, where reference is made to a specific cannabinoid, the cannabinoid can be in its acid, its non-acid form, or be a mixture of both acid and non-acid forms. For example, when referring to a THC content, one refers to THC in its acid, its non-acid form, or be a mixture of both acid and non-acid forms.

In some embodiments, the hashish product of the present disclosure may include one or more cannabinoid(s), such as THC, CBD, CBG, CBN, or any combinations thereof, in similar or different amounts.

In some embodiments, the hashish product of the present disclosure contains the one or more cannabinoid(s) in an amount (the “cannabinoid content”) sufficient for the user to experience a desired effect when consuming the product. For example, the hashish product may comprise from about 5 wt. % to about 90 wt. % cannabinoid, for example up to about 60 wt. %, or up to about 50 wt. %, or up to about 40 wt. %, or up to about 30 wt. %. Preferably, the hashish product includes at least about 20 wt. %, or at least about 25 wt. % cannabinoid. Preferably, the cannabinoid content refers to THC content.

In one embodiment, the hashish product of the present disclosure may be shaped into a desired shape. For example, the isolated trichomes and water mixture may be pressed in such manner to impart a specific shape to the cohesive mass. For example, the mixture may be placed in a hollow mold to impart a desired shape to the hashish product. For example, such shape may be square-like, ovoid-like, tablet-like, ball-like, etc. The reader will readily understand that other techniques may be used to impart shapes to the cohesive mass without departing from the present disclosure.

The hashish product as described herein may be packaged following its production. A package containing the hashish product may also have a corresponding shape or any other suitable shape. For example, a package may have a tablet-like shape and may include several discrete hashish product units, each unit having the same or a different shape from one another. For example, each discrete hashish product unit may have a square shape.

In some embodiments, each discrete hashish product unit may have substantially the same cannabinoid content from one another, as may be required under local legislation and regulations. In a non-limiting example, a cannabis-related package may include a total of up to 1000 mg of THC and a single hashish product unit may include up to about 10 mg of THC. The reader will understand that other THC content may be included in each single hashish product unit or in a package containing a plurality of hashish product units, depending on existing local legislation or regulations or changes made thereto.

In some embodiments, each discrete hashish product unit can have a predictable weight, which may be determined by the intended use of the product or by the number of units that are included in a package. For example, and without being limiting, each unit of the hashish product may have a weight of from about 0.2 g to about 20.0 g, including any ranges there in-between or any values therein. For example, the hashish product may have a total weight of 0.2 g, about 0.5 g, about 1.0 g, about 1.5 g, about 20.0 g, about 20.5 g, about 3.0 g, about 3.5 g, about 4.0 g, about 4.5 g, about 5.0 g, about 5.5 g, about 6.0 g, about 6.5 g, about 7.0 g, about 7.5 g, about 8.0 g, about 8.5 g, about 9.0 g, about 9.5 g, about 10.0 g, about 10.5 g, about 11.0 g, about 11.5 g, about 120.0 g, about 120.5 g, about 13.0 g, about 13.5 g, about 14.0 g, about 14.5 g, about 15.0 g, about 15.5 g, about 16.0 g, about 16.5 g, about 17.0 g, about 17.5 g, about 18.0 g, about 18.5 g, about 19.0 g, about 19.5 g, or about 20.0 g. In some cases, a variability of about +/−10% on the measured weight can be acceptable to the manufacturer. For example, when a desired weight (e.g., as indicated on the consumer product package) is of 2.0 g, it may be acceptable to the manufacturer that the actual weight of a hashish product in that package is of 2.0 g+/−10%, i.e., from 1.8 g to 2.2 g.

### Manufacturing Hashish Products

The present disclosure relates to hashish products including a cohesive mass of isolated trichomes.

Various methods for making a hashish product are described, and broadly include providing isolated cannabis trichomes, mixing with water and pressing under conditions sufficient to obtain the cohesive mass of isolated trichomes.

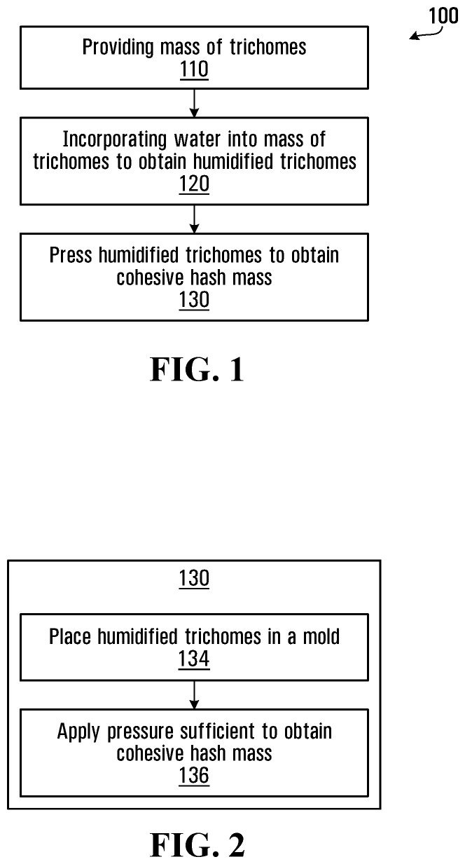

FIG. 1 is a flow chart of a general process 100 of making a hashish product in accordance with an embodiment of the present disclosure. The process 100 includes providing isolated cannabis trichomes at step 110. The trichomes may have been isolated from a single cannabis plant strain or from a plurality of cannabis plant strains that may have different respective cannabinoid concentrations and/or terpene blends. Both are viable options, the choice of one over the other being driven by practical considerations, such as inventory management considerations and/or cannabinoid content of the hashish product. The isolated cannabis trichomes are typically kief.

In one non-limiting embodiment, the isolated trichomes at step 110 can be obtained from a dry-sifting process (i.e., “dry-sifted” kief), a dry-ice batch process, or any other suitable method known in the art. If desired, the mass of trichomes may be cured/dried under conditions enough to decarboxylate at least a portion of the cannabinoids contained therein. For example, the mass of isolated trichomes may be placed in an oven at a temperature of about 225° F. for a time period of about 45 minutes, then cooled for at least 30 minutes at room temperature. Other decarboxylation conditions are known in the art and will not be further described in detail here for conciseness' sake.

At step 120, water is incorporated into the mass of isolated trichomes which is then mixed to obtain a mixture of humidified trichomes. Water can be incorporated in the form of steam, liquid or in combination, and may be distilled, reverse osmosis and/or microfiltered water. The relative amount of water being incorporated into the mass of trichomes at step 120 may dependent on a number of factors, such as desired maximal kief dilution (which in turn may affect the final THC content), kief grade (which in turn may impact the desired dilution), pressing conditions (such as pressure and/or pressing time) and heating temperature. For example, the relative amount of water being incorporated into the mass of trichomes at step 120 can be such that the final THC content is higher than the THC content typically contained in cannabis buds (which is typically of about 20 wt. % or less). In general, the amount of water to incorporate into the mass of trichomes is such that V_{T}/V_{W}>1, where V_{T} represents the trichomes volume (or mass) and V_{W} the water volume (or mass). For example, the amount of water to incorporate into the mass of trichomes can be such that V_{T}/V_{W} is from >1 to about 20, including any ranges there in-between or any values therein. For example, V_{T}/V_{W} can be of from about 5 to about 15, or from about 7 to about 15. For example, V_{T}/V_{W} can be of about 2, about 3, about 4, about 5, about 6, about 7, about 8, about 9, about 10, about 11, or about 12, about 13, about 14, about 15, or about 16. Alternatively, or additionally, the relative amount of water being incorporated into the mass of trichomes at step 120 can be such that the moisture content in the mixture is of at least 10 wt. %. Without being bound by any particular theory, it is believed that this step of incorporating water into the isolated cannabis trichomes allows the use of various grades of kief or mixtures thereof—the added water operating to increasing the total moisture present in the kief thus obtaining higher grade kief behavior during the pressing and/or hashish manufacturing subsequent steps.

At step 130, the humidified trichomes are pressed to obtain a cohesive mass of the isolated trichomes (or “pressed hashish”). Such pressing may be performed manually or mechanically, for example. In some embodiments, steps 120 and 130 may be performed sequentially or concurrently, for example. Steps 120 and 130 are performed under conditions enough to obtain a cohesive mass of the isolated trichomes.

For example, the conditions to obtain a cohesive mass of the isolated trichomes may include temperature, mixing speed, and pressure, which may be varied to alter the characteristics of the hashish product. The characteristics of the cohesive mass of isolated trichomes that are altered may include, but without being limited to, ductility (i.e., characteristic that defines the level of malleability of the hashish product), hardness or resistance to localized deformation (i.e., characteristic that determines how easy it is to cut or separate the hashish product), toughness (i.e., characteristic that determines the likelihood that the hashish product deforms rather than fractures under an applied force), color, tactual characteristics, and the like.

In some examples, the conditions may include submitting the isolated trichomes and water mixture to a thermal treatment (heating) to a temperature of less than 120° C. For example, less than 90° C., or less than 85° C., or at least 80° C. For example, a temperature of from about 30° C. to about 120° C., such as about 40° C. or 60° C.

In some examples, the conditions may include submitting the humidified trichomes mixture to a pressure at values of from about 0.5 to about 15 US tons, including any ranges therein or any value therein. For example, the pressure being applied to the humidified trichomes may include a pressure in the range of about 1 to about 15 US tons, or about 1 to about 10, or about 2 to about 8 US tons, or any value within such ranges. For example, the pressure may be of about 1 US ton, about 2 US tons, about 3 US tons, about 4 US tons, about 5 US tons, about 6 US tons, about 7 US tons, about 8 US tons, about 9 US tons, about 10 US tons, about 11 US tons, about 12 US tons, about 13 US tons, about 14 US tons, or about 15 US tons. Additionally, in a non-limiting example, the pressure being applied to the humidified trichomes may be performed for a time period ranging from about 0.5 (30 seconds) to about 5 minutes, or from about 1 to about 3 minutes, or from about 1 to about 2 minutes, or any value within such ranges.

In some embodiments, the conditions may include mixing the isolated trichomes and water at a value of for example from 15 to 1000 rpm, such as from 20 to 500 rpm, or from 25 to 450 rpm, or from 30 to 400 rpm, or from 45 to 450 rpm including any value within any of these ranges.

The reader will readily understand that in some embodiments, the conditions include applying the pressure while mixing and/or heating.

In one non-limiting embodiment as shown in FIG. 2, the humidified trichomes can be pressed at step 130 to obtain the cohesive mass. For example, the humidified trichomes can be placed into a hollow mold at step 134 and then applying pressure onto the humidified trichomes enough to obtain the cohesive mass at step 136. The reader will appreciate that other suitable techniques may be used instead of the mold/press described here, without departing from the disclosure. Advantageously, when using a hollow mold at step 134, the person of skill may elect to use a mold having a desirable shape and/or dimensions, required for commercializing a unit of the hashish product. The person of skill will readily appreciate that the amount and duration of the pressure being applied to the humidified trichomes may be determined based on various factors such as, volume/mass of humidified trichomes, moisture content of the humidified trichomes, desired textural consistency, pliability and/or crumbliness of the resulting hashish product. If desired, the pressing step 130 can be performed once or twice, depending on the characteristics of the humidified trichomes. For example, these characteristics may depend on the starting trichome strain, which may have variable wax contents, thus requiring one or more pressing steps to obtain the desired cohesive mass.

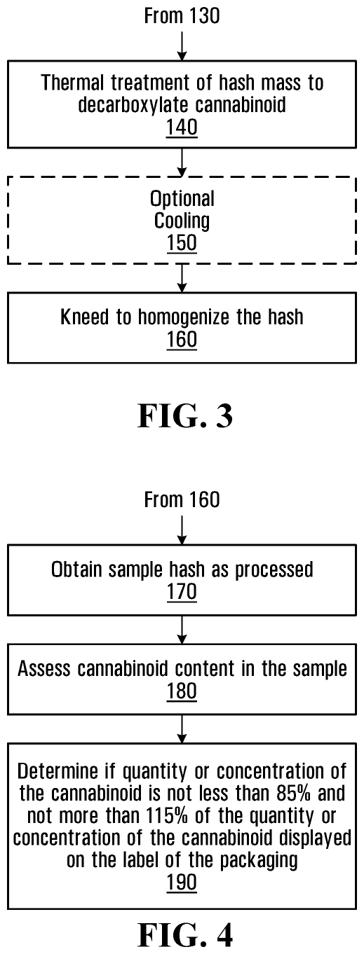

In one non-limiting embodiment as shown in FIG. 3, the pressed hashish product may be further processed, for example, in an optional additional thermal treatment in step 140 to initiate (or complete previously initiated) decarboxylation of cannabinoids contained in the pressed hashish product. This thermal treatment step 140 can be performed under conditions enough to decarboxylate at least a portion of the cannabinoids contained in the hashish product while preferably maintaining a value of at least 10 wt. % of water in the hashish product. For example, the thermal treatment step 140 can be performed at a temperature enough to achieve the desired result, such as less than 90° C., or less than 85° C., such as 80-85° C., or any value within such range. For example, the thermal treatment step 140 can be performed for a period of time sufficient to achieve the desired result, such as less than 40 minutes, or less than 30 minutes, such as 10 to 40 minutes, or any time period within such range.

In some embodiments, steps 130 and 140 may be performed sequentially or concurrently, for example. For example, in the embodiment where step 130 involves using a press and a mold, the press may have plates that are heated, thus performing steps 130 and 140 concurrently. Other suitable implementations of sequential or concurrent application of these steps are also possible.

In one non-limiting embodiment, the thermal treatment of step 140 can be followed with an optional cooling step 150, for example at room temperature.

If desired, the hashish product may be further processed at an optional kneading step 160 to ensure a more complete distribution of the contents within the hashish product, such as for example water, aroma, etc. Such kneading step 160 may be performed using any suitable device, such as an automated batch kneading system available from VMI (France).

To perform a quality test for the hashish product, the cannabinoid content from a sample of the as-produced hashish product from any one of steps 130, 140, 150, 160 can be determined as shown in FIG. 4. For example, the quality test may include obtaining a sample of the as-produced hashish product at a step 170. At a step 180, the cannabinoid content is determined using any suitable technique known in the art, such as for example Gas Chromatography/Mass Spectrometry (GC/MS), High Pressure Liquid Chromatography (HPLC), Gas Chromatography/Flame Ionization Detection (GC/FID), and the like. At a step 190, the criteria for quality assessment may include ensuring that the cannabinoid content in a hashish product acceptable under local legislation or regulations. In a non-limiting example, legislation/regulation may require that the cannabinoid content in a hashish product must not be less than 85% and no more than 115% of the cannabinoid content displayed on the packaging label.

### EXAMPLES

The following examples describe some exemplary modes of making and practicing certain compositions that are described herein. These examples are for illustrative purposes only and are not meant to limit the scope of the compositions and methods described herein.

### Example 1—Segmentation Test

The textural consistency, pliability and/or crumbliness of the herein described hashish product can be assessed using a segmentation test as described hereinafter. In this test, a hashish product is segmented along an axis using a cutting blade and the amount of residual product determined thereafter.

The test procedure is as follows:
a) 100 hashish product samples to be simultaneously tested (herein referred as “test samples”), which are all made in a single batch or individually but in a sufficiently controlled environment such as to ensure a high degree of uniformity between the samples are provided.
b) The test samples are conditioned for 1 h at a temperature of 20° C. and at a humidity level of 40%.
c) Each test sample is tested by placing same on a support surface. For test samples that are not spherical, the test samples are placed on the support surface in an orientation such that the same side of the test samples will face up, if applicable. A single blade is then used to slice the test sample along a single line to obtain substantially two identical segments.
d) Each segment is then weighted on an analytical balance, such as a Mettler Toledo™ NewClassic ME Analytical Balances (Fisher Scientific, USA), and the amount of loss material is reported for each segment as per the following ratio S_{W}/E_{W} where S_{W} represents the segment weight and E_{W} represents the expected weight. A ratio of 0.90 or less is considered a failure; loss of at least 10 wt. % hashish indicates a failure of the test.
e) Each test sample is classified into respective pass/fail groups based on the ratio determined for the respective pair of segments. The probability of failure per single hashish product sample failure is computed by dividing the number of pellets that have failed by 100, which is the total number of test samples.

Note that for the purpose of the present description, the above defined test procedure will be referred to as a “segmentation test”.

According to the present disclosure, the probability of failure per hashish product does not exceed 0.25, or does not exceed 0.20, or does not exceed 0.15, or does not exceed 0.10.

### Example 2—Moisture Content Distribution Test

The moisture content distribution in the hashish product can be assessed using a moisture content distribution test as described hereinafter. In this test, a hashish product is segmented using a cutting blade to obtain a first portion and a second portion and the moisture content is determined thereafter.

Moisture analyzers are used for rapid determination of total moisture of a sample using the thermogravimetric method. Moisture of a material includes all volatile components of a sample which may be released by heating, this includes water, alcohols, oils, volatile metabolites (terpenes, cannabinoids), substance produced through thermal degradation (e.g. by overheating), and others.

The thermogravimetric method (an evolution of loss on drying) determines moisture by measuring the mass of the sample while heating, a moisture balance will continuously monitor the mass of the sample during heating until there is no longer a change in mass at which point it will automatically stop the measurement and display the calculated moisture content. As the Moisture balance measures all volatiles care must be taken not to overheat or char samples in order to ensure reproducible results.

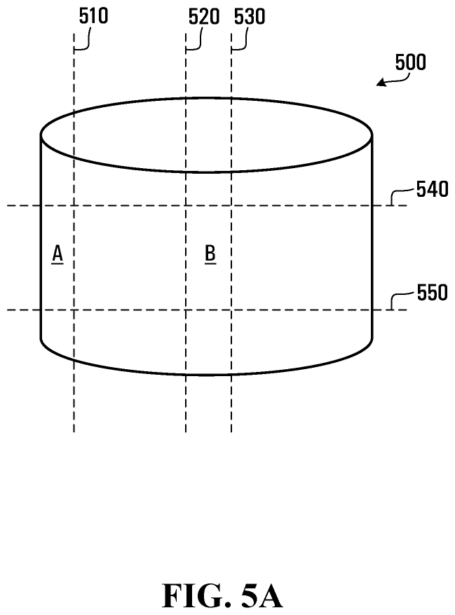

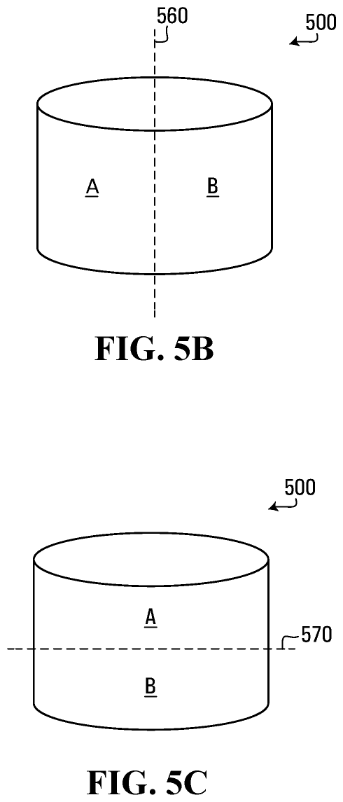

The test procedure is as follows:
a) A predetermined number of hashish product samples to be simultaneously tested (herein referred as “test samples”), which are all made in a single batch or individually but in a sufficiently controlled environment such as to ensure a high degree of uniformity between the samples are provided.
b) The test samples are conditioned for 1 h at a temperature of 20° C. and at a humidity level of 40%.
c) Each test sample is tested by placing same on a support surface. For test samples that are not spherical, the test samples are placed on the support surface in an orientation such that the same side of the test samples will face up, if applicable. A single blade can then be used to slice the test sample to obtain at least two portions thereof.
   i. For example, in embodiments where the test sample is voluminous enough (e.g., may include 2 g or more of mass and/or may have a size of sufficient dimensions), the single blade can be used to slice test sample 500 along two 2 lines 520, 530 along a longitudinal axis thereof, substantially parallel to each other, as shown in FIG. 5A. The single blade can then be used to slice the test sample 500 along two 2 lines 540, 550 along a transverse axis thereof, substantially parallel to each other. The single blade can then be used to slice the test sample 500 along 1 line 510 substantially parallel to lines 520, 530, and closer to the outer edge of test sample 500. The crossing of axes 520, 530 with 540, 550 produce a second portion B whereas the crossing of axes 540, 550 with 510 produces a first portion A. In this embodiment, while the first portion is shown as a peripheral portion and the second portion is shown as a core portion, the person of skill will readily understand that portions from other subsections of the test sample are also possible.
   ii. For example, in embodiments where the test sample 500 is smaller in mass or size than the previous embodiment, the single blade can be used to slice the test sample 500 along a longitudinal line 560 to obtain a first portion A and a second portion B, as shown in FIG. 5B. Alternatively, the single blade can be used to slice the test sample 500 along a transverse line 570 to obtain a first portion A and a second portion B, as shown in FIG. 5C.
d) The moisture content of each of the first and second portions A and B is then determined, for example using USP NF 731 Loss On Drying method.

The moisture content of various portions from the same test sample can be obtained as per variations of the above-described procedure in order to determine the moisture content a various location in the test sample and, thus, determine the moisture content distribution in the test sample.

Alternatively, the moisture content in a whole test sample can be assessed and the value compared to a second moisture content from another sample originating from the same batch of hashish products and/or from a pre-determined moisture content reference.

Note that for the purpose of the present description, the above defined test procedure will be referred to as a “moisture content distribution test”.

### Example 3

In this example, a hashish product was made in accordance with an embodiment of the present disclosure.

A volume of 10.0 ml of distilled water was added to a mass of 40.0 g of dried trichomes from strain Cxwwb and mixed thoroughly. The humidified mixture of trichomes was placed into a clean mold and pressed with 3 US tons of pressure for 2 minutes to obtain a cohesive mass of isolated trichomes. The pressing was obtained with 7.5″ by 8.5″ pressing plate having a pressing area of 63.75 inches squared (such that a pressure of 5 tons corresponds to about 156 psi, and a pressure of 10 tons corresponds to about 313 psi).

The cohesive mass of pressed trichomes was placed in a bag, which was then sealed and submerged in a heated water bath having a temperature between 80-85° C. The bag was left with an opening protruding from the water to vent CO₂ resulting from the decarboxylation of the hashish product. The temperature of the water bath was maintained between 80° C. and 85° C. while heating the product for 40 minutes.

The hashish product was then allowed to cool at room temperature for 10 minutes, and then kneaded to homogenize its contents.

The resulting hashish product contained <30% THC, 19.6 wt. % moisture content, and had a satisfactory consistency, texture and appearance.

### Example 4

In this example, a hashish product was made as per Example 3 but with cannabis strain SL, using a volume of 5.00 ml distilled water with 40.0 g dried trichomes, with 5 US tons of pressure for 1 min followed by a second identical press. The bag was incubated in the heated water bath having a temperature between 80-85° C. for 30 minutes.

The resulting hashish product had a satisfactory consistency, texture and appearance.

### Example 5

In this example, a hashish product was made as per Example 3 but with a different pressing load value, only one pressing step, and varying water volume incorporated. The parameters used are reproduced in the following table 1.

TABLE 1

| | | | | | Water | Water | | |
|---|---|---|---|---|---|---|---|---|
| | Mass | Mass | Pressure | Pressure | Bath | Bath | Cooling | Moisture |
| | Kief | Water | (US | Time | Temp. | Time | Time | content |
| Strain | (g) | (g) | Tons) | (min) | (C.) | (min) | (min) | (wt. %) |
| CxWWb | 26.00 | 3.25 | 3 | 2 | 83.8 | 40 | 10 | 16.1 |
| NLxBB | 30.00 | 3.75 | 3 | 1 | 83.8 | 40 | 10 | 19.6 |
| KSa | 30.01 | 3.74 | 2 | 1 | 82.65 | 40 | 10 | 16.5 |
| Ksa | 30.00 | 3.75 | 2 | 1 | 82.65 | 40 | 10 | 16.7 |
| Ksa | 30.01 | 3.74 | 2 | 1 | 83.65 | 44 | 10 | 19.4 |
| Ksa | 30.02 | 3.75 | 2 | 1 | 83.65 | 44 | 10 | 18.5 |
| Ksa | 30.00 | 3.77 | 2 | 1 | 83.5 | 40 | 10 | 19.2 |
| Ksa | 30.00 | 3.77 | 2 | 1 | 83.5 | 40 | 10 | 16.6 |
| Ksa | 30.01 | 3.75 | 2 | 1 | 83.5 | 40 | 10 | 17.0 |
| Ksa | 30.00 | 3.74 | 4 | 1 | 83.5 | 40 | 10 | 18.7 |

The resulting hashish products had a satisfactory consistency, texture and appearance.

### Example 6

In this example, a hashish product was made as per Example 3 but with a different pressing load value, only one pressing step, and varying water volume incorporated. The parameters used are reproduced in the following table 2.

TABLE 2

| | | | | | Water | Water | | |
|---|---|---|---|---|---|---|---|---|
| | Mass | Mass | Pressure | Pressure | Bath | Bath | Cooling | Moisture |
| | Kief | Water | (US | Time | Temp. | Time | Time | content |
| Strain | (g) | (g) | Ton) | (min) | (C.) | (min) | (min) | (wt. %) |
| KSa | 60 | 0 | 1 | 1 | 88.9 | 40 | 10 | 0 |
| KSa* | 60 | 7.5 | 1 | 1 | 88.9 | 40 | 10 | 24.5 |
| KSa* | 40 | 4.0 | 1 | 1 | 89 | 43 | 10 | 15.9 |
| KSa* | 40 | 3.5 | 1 | 1 | 88.2 | 40 | 10 | 18.3 |
| KSa* | 40 | 3.6 | 1 | 1 | 88.6 | 40 | 10 | 17.3 |

The resulting hashish products marked with “*” had a satisfactory consistency, texture and appearance. The hashish product without “*” had unsatisfactory consistency, texture and appearance.

### Example 7

In this example, a hashish product was made as per Example 3 but with different pressing load value, only one pressing step, and varying water volume incorporated. The parameters used are reproduced in the following table 3.

TABLE 3

| | | | | | Water | Water | |
|---|---|---|---|---|---|---|---|
| | Mass | Mass | Pressure | Pressure | Bath | Bath | Cooling |
| | Kief | Water | (US | Time | Temp. | Time | Time |
| Strain | (g) | (g) | Ton) | (min) | (C.) | (min) | (min) |
| KSa-NLxxBB | 40 | 5.0 | 1 | 5 | 80 | 40 | 10 |
| KSa-NLxxBB | 40 | 5.0 | 1 | 5 | 80 | 40 | 10 |
| KSa-NLxxBB | 40 | 5.0 | 1 | 3 | 80 | 43 | 15 |
| KSa-NLxxBB | 40 | 5.0 | 1 | 3 | 80 | 40 | 15 |
| KSa-NLxxBB | 40 | 5.0 | 1 | 3 | 80 | 33 | 2 |

The resulting hashish products had a satisfactory consistency, texture and appearance (very dark in this case).

### Example 8

In this example, a hashish product was made as per Example 3 but with different pressing load value, only one pressing step, and varying water volume incorporated. The parameters used are reproduced in the following table 4.

TABLE 4

| | | | | | Water | Water | | |
|---|---|---|---|---|---|---|---|---|
| | Mass | Mass | Pressure | Pressure | Bath | Bath | Cooling | Moisture |
| | Kief | Water | (US | Time | Temp. | Time | Time | content |
| Strain | (g) | (g) | Tons) | (min) | (C.) | (mm) | (min) | (wt. %) |
| NLxxBB | 40 | 5.04 | 1 | 1 | 80 | 40 | 10 | 18.7 |
| NLxxBB | 40 | 5 | 1 | 5 | 80 | 60 | 10 | 16.4 |
| NLxxBB | 40 | 5 | 1 | 5 | 80 | 50 | 5 | 7.6 |
| NLxxBB | 40 | 5 | 1.5 | 5 | 80 | 60 | 5 | 13.5 |
| NLxxBB | 40 | 5 | 2 | 5 | 80 | 50 | 10 | 16.3 |
| NLxxBB | 40 | 5 | 2 | 1 | 80 | 50 | 10 | 11 |
| NLxxBB | 40 | 5 | 5 | 2 | 80 | 60 | 10 | 10.7 |
| NLxxBB | 40 | 5 | 10 | 2 | 80 | 40 | 0 | 7.5 |
| NLxxBB | 40 | 5 | 1 | 0.5 | 80 | 45 | 5 | 9.2 |
| NLxxBB | 40 | 5 | 0.3 | 1 | 80 | 45 | 5 | 15 |
| NLxxBB | 40 | 5 | 1 | 0.5 | 80 | 40 | 10 | 16.3 |
| NLxxBB | 40 | 5 | 1 | 1 | 80 | 35 | 5 | 16.3 |
| NLxxBB | 40 | 10 | 1 | 1 | 80 | 40 | 1 | 34.8 |
| NLxxBB | 40 | 7.5 | 1 | 1 | 80 | 50 | 10 | 18.5 |
| NLxxBB | 40 | 5 | 1 | 1 | 80 | 50 | 0 | 19.7 |

The resulting hashish product had a satisfactory consistency, texture and appearance.

### Example 9

In this example, a hashish product was made as per Example 3 by mixing 88 wt. % KSa kief and 11 wt. % water.

The kief moisture content and water content in table 5 below were measured as follows: loss on drying technique using the moisture analyzer MA160 from Sartorius (water content), and Chilled mirror dew point technique using the capacitive hygrometer Aqualab 4TE from Meter Group (water activity). These measurements were performed for the kief prior to incorporating any water, after incorporating water and mixing, after pressing to obtain a cohesive mass of trichomes, as well as 4 weeks later to test for stability of the moisture content/water activity in the hashish product. The results are produced in table 5 below.

TABLE 5

| | | |
|---|---|---|
| | Moisture content | |
| Test | (wt. %) | Water activity |

Isolated trichomes from Ksa strain before mixing with water

| | | | |
|---|---|---|---|
| 1 | 13.50% | 0.5375 | 25.45° C. |
| 2 | 12.91% | 0.5329 | 25.15° C. |
| 3 | 12.59% | 0.5349 | 25.09° C. |

Isolated trichomes and water after mixing

| | | | |
|---|---|---|---|
| 1 | 19.85% | 0.8611 | 25.12° C. |
| 2 | 18.96% | 0.8593 | 25.16° C. |
| 3 | 19.49% | 0.8752 | 25.16° C. |

Hash product

| | | | |
|---|---|---|---|
| 1 | 16.78% | 0.894 | 25.12° C. |
| 2 | 14.27% | 0.8946 | 25.13° C. |
| 3 | 15.08% | 0.8962 | 25.12° C. |

Hash product after 4 days

| | | | |
|---|---|---|---|
| 1 | 14.85% | 0.8708 | 25.19° C. |
| 2 | 13.80% | 0.8684 | 25.13° C. |
| 3 | 14.08% | 0.8657 | 25.08° C. |

### Example 10

In this example, several hashish product batches were made using a single NLxBB kief batch following the procedure set out in Example 3 (including the water bath treatment at 80° C. for 40 minutes in each case) and the specific parameters set out in table 6. The pressing, however, was obtained with a first 2″ by 4″ pressing plate having a pressing area of 8 inches squared (such that a pressure of 3.9 tons corresponds to about 975 psi, a pressure of 5 tons corresponds to about 1250 psi, and a pressure of 10 tons corresponds to about 2500 psi) and a second 2.5″ by 2.5 pressing plate having a pressing area of 6.25 inches squared (such that a pressure of 3.9 tons corresponds to about 1248 psi, a pressure of 5 tons corresponds to about 1600 psi, and a pressure of 10 tons corresponds to about 3200 psi). The kief initially contained about 9% moisture content, 0.65 water activity (a_{w}), about 360 mg/g THC (36 wt. %).

For each lot, the mass of kief was mixed with the mass of water by hand for a minimum of 5 minutes. For samples pressed with the 2″ by 4″ plate, 20 g was loaded for each brick, for a total of 3 bricks each lot. For the samples pressed with the 2.5″ by 2.5″ plate, 15.5 g was loaded for each brick, for a total of 1 brick each lot. The reported yield % refers to the percentage of the weight of hashish recovered after the pressing relative to the combined input weight of kief and water used for making the hashish.

TABLE 6

| | Moisture | | | 1^{st} | 1^{st} | 2^{nd} | 2^{nd} | |
|---|---|---|---|---|---|---|---|---|
| | content in | Mass | Mass | Pressure | Pressure | Pressure | Pressure | |
| | mixture | Kief | Water | (US | Time | (US | Time | Yield |
| Lot # | (%) | (g) | (g) | Tons) | (min) | Tons) | (min) | (%) |
| BBH-082 | 10 | 60 | 0.61 | 5 | 5 | 5 | 2 | 94.02 |
| BBH-083 | 10 | 60 | 0.61 | 10 | 5 | 10 | 2 | 77.23 |
| BBH-084 | 15 | 60 | 4.17 | 5 | 5 | 5 | 2 | 77.92 |
| BBH-085 | 15 | 65 | 4.52 | 10 | 5 | 10 | 2 | 40.14 |
| BBH-086 | 20 | 65 | 8.03 | 5 | 5 | 5 | 2 | 57.48 |
| BBH-087 | 20 | 65 | 8.03 | 10 | 5 | 10 | 2 | 42.12 |
| BBH-088 | 35 | 15 | 5.27 | 3.9 | 5 | 3.9 | 2 | 58.47 |
| BBH-089 | 59 | 15 | 15 | 3.9 | 5 | 3.9 | 2 | 38.33 |
| BBH-090 | 84 | 5 | 15 | 3.9 | 5 | 3.9 | 2 | 39.20 |

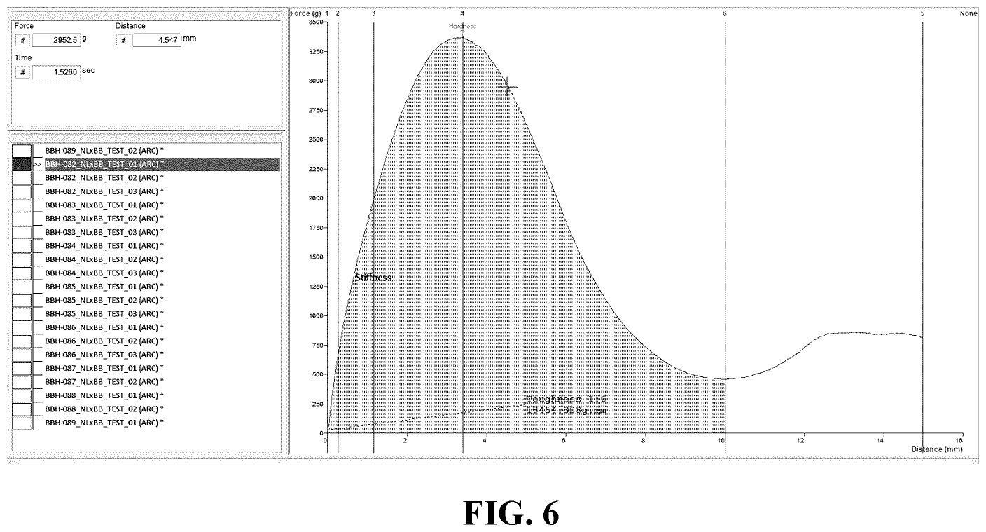

A lot included three bricks of hashish products. A 20 mm by 20 mm portion was cut from each brick produced and submitted to a three-point bend test. A probe attached to a 100 kg load cell was lowered, and both the distance travelled and force exerted on the probe were measured as the hash portion was put under stress. A force over displacement graph was generated by the texture analyzer software (see for example FIG. 6), and a macro was used to calculate the slope of the rise (before breaking point), maximum force, and total area under the graph. These values in order are represented as stiffness (g/mm), breaking point (g), and toughness (g*mm) in the following table 7.

TABLE 7

| | | | |
|---|---|---|---|
| | | | Average |
| Test 1 | Test 2 | Test 3 | (standard deviation) |

BBH-082

| | | | | | |
|---|---|---|---|---|---|
| Stiffness (g/mm) | 1480 | 847 | 877 | 1068 | (291) |
| Breaking point (g) | 3370 | 1958 | 1517 | 2282 | (790) |
| Toughness(g*mm) | 1844 | 11042 | 6051 | 11849 | (5096) |

BBH-083

| | | | | | |
|---|---|---|---|---|---|
| Stiffness (g/mm) | 570 | 1133 | 634 | 779 | (252) |
| Breaking point (g) | 817 | 2378 | 992 | 1396 | (698) |
| Toughness (g*mm) | 3122 | 13726 | 3996 | 6948 | (4806) |

BBH-084

| | | | | | |
|---|---|---|---|---|---|
| Stiffness (g/mm) | 1253 | 314 | 1023 | 863 | (400) |
| Breaking point (g) | 2090 | 614 | 1448 | 1384 | (604) |
| Toughness (g*mm) | 11589 | 3700 | 6185 | 7148 | (3304) |

BBH-085

| | | | | | |
|---|---|---|---|---|---|
| Stiffness (g/mm) | 369 | ND | 170 | 269.5 | (99.5) |
| Breaking point (g) | 576 | ND | 256 | 416 | (160) |
| Toughness (g*mm) | 2630 | ND | 952 | 1791 | (839) |

BBH-086

| | | | | | |
|---|---|---|---|---|---|
| Stiffness (g/mm) | 263 | 184 | ND | 223.5 | (39.5) |
| Breaking point (g) | 514 | 379 | ND | 446.5 | (67.5) |
| Toughness (g*mm) | 3140 | 2111 | ND | 2625.5 | (514.5) |

The present inventors have also obtained hashish products with the herein described procedure that are characterized with one or more of a breaking point of from about 250 g to about 8000 g, a stiffness of from about 200 g/mm to about 8500 g/mm, and a toughness of from about 1500 g*mm to about 12,500 g*mm.

The density of core and peripheral portions of hashish products from lots BBH-082 to BBH-087 was measured using water displacement method with a weigh scale and water filled vessel and the results are reported in the following table 8.

For each brick with sufficient yield, a portion of the core hash material was isolated using a knife. A periphery portion of each of these bricks was also isolated using a knife. These sample were weighed to determine the mass of the sample, and then the volume was determined using water displacement method. Density was calculated from these values as mass/volume.

TABLE 8

| | Density core portion (g/ml) | | | | Density peripheral portion (g/ml) | | | |
|---|---|---|---|---|---|---|---|---|
| | | | | Average | | | | Average |
| Lot # | Test 1 | Test 2 | Test 3 | (St. Dev.) | Test 1 | Test 2 | Test 3 | (St. Dev.) |
| BBH-082 | 0.316 | 0.338 | 0.313 | 0.322 (0.011) | 0.515 | 0.588 | 0.432 | 0.511 (0.064) |
| BBH-083 | 0.405 | 0.366 | 0.355 | 0.375 (0.021) | 0.435 | 0.554 | 0.387 | 0.458 (0.070) |
| BBH-084 | 0.554 | 0.579 | 0.425 | 0.519 (0.067) | 0.567 | 0.704 | 0.617 | 0.629 (0.057) |
| BBH-085 | 0.496 | 0.387 | 0.324 | 0.402 (0.071) | 0.453 | 0.411 | 0.588 | 0.484 (0.076) |
| BBH-086 | 0.371 | 0.556 | 0.395 | 0.441 (0.082) | 0.429 | 0.680 | 0.504 | 0.538 (0.105) |
| BBH-087 | 0.530 | 0.370 | 0.456 | 0.452 (0.065) | 0.448 | 0.713 | 0.441 | 0.534 (0.127) |

The water activity (a_{w}) and total THC content (THC and THCA content measured with HPLC) in a sample from each lot was measured with a Chilled Mirror Dew Point Hygrometer and the results are reported in the following table 9.

TABLE 9

| | Water activity | Total THC | | |
|---|---|---|---|---|
| Lot # | (a_{w}) | (wt. %) | THC | THCA |
| BBH-082 | 0.4415 | 31.65 | 118.6 | 225.7 |
| BBH-083 | 0.4629 | 32.47 | 125.6 | 227.0 |
| BBH-084 | 0.6972 | 32.15 | 166.1 | 177.3 |
| BBH-085 | 0.6485 | 32.75 | 263.4 | 73.1 |
| BBH-086 | 0.8455 | 30.36 | 247.9 | 63.4 |
| BBH-087 | 0.8118 | 30.91 | 248.7 | 68.9 |
| BBH-088 | 0.9204 | 28.57 | 272.4 | 15.2 |
| BBH-089 | 0.9704 | 26.53 | 258.1 | 8.2 |
| BBH-090 | 0.9660 | 27.81 | 273.2 | 5.6 |

The present inventors have determined that moisture content is directly related for pliability, % decarboxylation, and helps with heat conductivity. The present inventors also have determined that bacterial growth is not directly tied to moisture content, but how the moisture content is bound by the product, this is where a water activity lower than 0.7 is preferable in specific implementations to increase shelf-life and avoid formation of mold over time. The present inventors also have determined that higher water activity shows greater % of decarboxylation of the cannabinoid(s), as shown with THC content vs. THC-A content in table 9, which can be useful to optimize the potency efficiency and improve user experience.

Other examples of implementations will become apparent to the reader in view of the teachings of the present description and as such, will not be further described here.

Note that titles or subtitles may be used throughout the present disclosure for convenience of a reader, but in no way should these limit the scope of the invention. Moreover, certain theories may be proposed and disclosed herein; however, in no way they, whether they are right or wrong, should limit the scope of the invention so long as the invention is practiced according to the present disclosure without regard for any particular theory or scheme of action.

All references cited throughout the specification are hereby incorporated by reference in their entirety for all purposes.

It will be understood by those of skill in the art that throughout the present specification, the term “a” used before a term encompasses embodiments containing one or more to what the term refers. It will also be understood by those of skill in the art that throughout the present specification, the term “comprising”, which is synonymous with “including,” “containing,” or “characterized by,” is inclusive or open-ended and does not exclude additional, un-recited elements or method steps.

As used in the present disclosure, the terms “around”, “about” or “approximately” shall generally mean within the error margin generally accepted in the art. Hence, numerical quantities given herein generally include such error margin such that the terms “around”, “about” or “approximately” can be inferred if not expressly stated.

Although various embodiments of the disclosure have been described and illustrated, it will be apparent to those skilled in the art considering the present description that numerous modifications and variations can be made. The scope of the invention is defined more particularly in the appended claims.

1. A hashish product, comprising a cohesive mass of isolated cannabis trichomes and at least 5 wt. % moisture content, wherein the moisture content is distributed throughout the cohesive mass to be detectable in at least 80 vol. % of the hashish product.

2. (canceled)

3. (canceled)

4. The hashish product according to claim 1, comprising at least 10 wt. % moisture content.

5. The hashish product according to claim 1, comprising less than 50 wt. % moisture content.

6. The hashish product according to claim 5, comprising less than 40 wt. % moisture content.

7. (canceled)

8. The hashish product according to claim 5, comprising less than 30 wt. % moisture content.

9. (canceled)

10. The hashish product according to claim 1, comprising less than 1000 ppm of an organic solvent, including 0.

11. (canceled)

12. The hashish product according to claim 1, wherein the moisture content is detectable in at least 90 vol. % of the hashish product.

13. The hashish product according to claim 1, wherein the hashish product has a weight of from about 0.2 g to about 20 g.

14. (canceled)

15. The hashish product according to claim 1, comprising a cannabinoid content of at least 20 wt. %.

16. (canceled)

17. The hashish product according to claim 1, wherein the isolated cannabis trichomes prior to forming the cohesive mass are kief.

18. The hashish product according to claim 17, wherein the kief is a dry-sifted kief.

19. The hashish product according to claim 17, wherein the kief is from a plurality of kief grades.

20. The hashish product according to claim 17, wherein the kief is a low grade kief comprising a tetrahydrocannabinol (THC) content of less than 40 wt. %.

21. The hashish product according to claim 1, wherein the isolated cannabis trichomes are from a plurality of strains of cannabis plants.

22. A process of making a hashish product, comprising
a) providing isolated cannabis trichomes;
b) incorporating water into the isolated cannabis trichomes and mixing same to obtain a mixture; and
c) pressing said mixture to obtain the hashish product, the hashish product comprising a cohesive mass of the isolated cannabis trichomes, wherein the water is distributed throughout the cohesive mass.

23. (canceled)

24. (canceled)

25. The process according to claim 22, wherein water at step b) is incorporated to obtain at least 5 wt. % moisture content in the hashish product.

26.-56. (canceled)

57. A hashish product made with the process according to claim 22.

58.-67. (canceled)

68. The hashish product according to claim 12, wherein the moisture content is detectable in at least 95 vol. % of the hashish product.

69. The hashish product according to claim 1, wherein the hashish product contains up to about 30 wt. % of tetrahydrocannabinol (THC).

70. The hashish product according to claim 1, wherein the hashish product has at least two of the following properties as determined in a three-point bending test: a breaking point of at least 250 g; a stiffness of less than 8,500 g/mm; and a toughness of less than 12,500 g*mm.

71. A hashish product, comprising a cohesive mass of isolated cannabis trichomes and at least 5 wt. % moisture content, wherein the isolated cannabis trichomes are from low grade kief comprising a tetrahydrocannabinol (THC) content of less than 40 wt. %.
